(19) United States
(12) Patent Application Publication
(10) Pub. No.: US 2012/0239006 A1
(43) Pub. Date: Sep. 20, 2012

(54) External control valve for indwelling urethral catheters

(76) Inventors: Bandula Wijay, (US); Omar Durrani, Houston, TX (US); Nandhika Wijay, Friendswood, TX (US)

(21) Appl. No.: 13/065,189

(22) Filed: Mar. 15, 2011

(51) Int. Cl. A61M 27/00

(52) U.S. Cl. 604/544

(57) ABSTRACT

An external control valve such as for an indwelling urethral catheter allows for selectively controlled urination via manual manipulation. The external valve is actuated by palpation as the patient compresses a proximal portion toward a distal portion of the valve. The valve includes a hollow stem having a bore in the wall thereof that is kept closed through the use of a biasing spring. Once the spring is compressed by the patient, bore is positioned within an opening in the distal portion, allowing urine to flow out of an opening in the proximal end of the valve.

### FIELD OF INVENTION

This invention relates to an external control valve for indwelling urethral catheters which enables patients to control urination while the indwelling catheter remains within the urethra.

### BACKGROUND OF THE INVENTION

Patients requiring urinary catheters for drainage of the bladder are typically given a Foley, or Foley-type, catheter which is inserted into the urethra and extends from the bladder to the exterior of the patient. In most cases, these urinary catheters, or indwelling catheters, are equipped with an inflatable balloon so that they can be anchored within the bladder of the patient. The portion of the catheter that extends externally from the patient is usually connected to a bag which collects the urine as it flows continually from the patient's bladder. The external or exterior portion of the catheter can also be sealed or clamped, if necessary. However, even though Foley, and Foley-type, catheters have been in widespread use for many decades, they still have certain drawbacks. For instance, these indwelling catheters can cause urinary tract infections, can leak, do not give the patient an ability to control urination, and restrict the patient's mobility or physical activities.

In order to address the drawbacks of Foley-type indwelling catheters, many different types of valves have been proposed. The main purpose of the valves described in the prior art is to provide the patient, especially the ambulatory patient, an ability to selectively control urination via manual manipulation. Valves can also eliminate the use of urine collection bags in ambulatory patients. Eliminating the need for collection bags is very beneficial because these bags do not give the user any control over urination, must be worn at all times, inhibiting the patient's activities, are inconvenient for the patient, and are a prime source for bacterial infection. There are two main types of valves for urinary indwelling catheters in the prior art: internal (those that are placed inside the patient's body) and external.

Internal urethral valves are placed within the urinary tract of the patient and are usually actuated by some type of external manipulation. There are four main types of internal valves in the prior art: valves that are actuated by (1) an externally extended cord or wire; (2) an externally positioned magnet; (3) externally applied palpitation; and (4) those having pressure dependent thresholds. In general, these valves are complex, expensive, difficult to implant, unreliable, susceptible to malfunction, and difficult to operate.

Rocchi et al., in U.S. Pat. No. 3,331,371, and Osthagen et al., in U.S. Pat. No. 3,503,400, describe an internally placed urethral valve which is actuated by manipulation of an externally extended cord or wire. The manually controlled internal valve is placed in the urethra near the bladder. The valve is opened by pulling on the cord or wire that extends through the urethra to the exterior of the patient. Although this design could enable the patient to control urination, this arrangement is highly susceptible to infection, causes discomfort, could become dislodged whenever the cord or wire is pulled with too much force, would be difficult to manipulate by older patients, requires an implantation procedure in order to properly place the valve within the patient, and is impractical as the patient's hand will be come covered in urine whenever the cord or wire is pulled.

A magnetically controlled valve could eliminate some of the drawbacks of a cord actuated internal valve, and there are many magnetically controlled internal urethral valves in the prior art. For example, Plishner, in U.S. Pat. No. 3,419,008, Osthagen et al., in U.S. Pat. No. 3,642,004, Loe, in U.S. Pat. No. 3,731,670, Isaacson, in U.S. Pat. No. 3,812,841, Roth, in U.S. Pat. No. 3,939,821, Bucalo, in U.S. Pat. No. 4,024,855, Barwick in U.S. Pat. No. 5,041,092, Ardito, in U.S. Pat. No. 5,140,999, Ardito, in U.S. Pat. No. 5,711,314, Iderstein et al., in U.S. Pat. No. 6,105,580, Whalen et al., in U.S. Pat. Nos. 6,527,702 and 7,001,327, and Flinchbaugh, in U.S. Pat. No. 6,855,126 all describe an internal urethral valve that is opened or closed utilizing an externally placed magnet. All of these inventions require the external placement of a magnet which will actuate an internally placed magnet which acts as, or is associated with, the internally placed valve. As with cord actuated valves, magnetically controlled valves are implanted within the urethra of the patient. Consequently, if the valve malfunctions, removal and additional implantation procedures will be required in order to replace the malfunctioning valve, adding to the cost and distress of the patient. Since the valve can only be actuated from an externally placed magnet, the patient must always have a magnet available in order to urinate. If the patient loses the magnet, or does not have one handy, a great deal of distress can occur for the patient. In addition, unintended discharge of urine can result if the patient unintentionally or accidentally comes in contact with a magnet or magnetic field.

Another type of internal urethral valve is one which opens whenever the patient applies force, such as squeezing, to the internally placed valve. These internally palpable valves open as the patient squeezes or compresses a bulb or a section of the device that acts as the valve. When the squeezing or applied pressure is released, the valve closes, allowing urine to collect again in the bladder. As with all of the other internal urethral valves, if the valve malfunctions and does not open or close properly, removal and additional implantation procedures are required, adding to the cost and distress of the patient. In addition, these valves typically require the patient to have good dexterity, which is often deficient in elderly patients, and the ability to locate and actuate the palpable valve or segment. Since the female urethra is quite short, these valves cannot typically be used on female patients. Internally palpable urethral valves are also unreliable, can leak, can be difficult to actuate, could damage the urethra and surrounding tissue, and may be uncomfortable for the patient. As with magnetically controlled internal urethral valves, there are many internally palpable urethral valves in the prior art. For example, Shulte, in U.S. Pat. No. 3,758, 073, Davis, in U.S. Pat. No. 4,350,161, Davis, in U.S. Pat. No. 4,432,757, Kos et al., in U.S. Pat. No. 4,643,169, Haber, in U.S. Pat. No. 4,846,784, Burton et al., in U.S. Pat. No. 4,909,785, Goldberg et al., in U.S. Pat. No. 4,932,938, Davis, in U.S. Pat. No. 4,946,449, Salama, in U.S. Pat. No. 4,968,294, and Burton et al., in U.S. Pat. No. 5,112,306 all describe an internal urethral valve actuated by externally applied palpitation.

Other internal urethral valves in the prior art are actuated whenever the fluid pressure reaches a certain threshold. These pressure dependent internal urethral valves are subject to leakage, do no not drain completely, and have predetermined pressure ranges that may not be appropriate for all patients and all situations. Typical pressure dependent internal urethral valve inventions are disclosed in Kwan-Gett et al., in U.S. Pat. No. 3,768,102, Kulisz et al., in U.S. Pat. No. 5,512,032, Kulisz et al., in U.S. Pat. No. 5,722,932, and Migachyov, in U.S. Pat. No. 6,183,413.

In order to eliminate the complexity, and other drawbacks, of existing internal urethral valves for indwelling urinary catheters, other inventors have designed exterior valves which can be attached to the portion of the indwelling urinary catheter that extends externally from the patient. As with internal urethral valves, the external urethral valves in the prior art can be categorized based upon their means of actuation, such as: magnetically, through palpitation, through rotation, and through pressure dependence. Barwick et al., in U.S. Pat No. 5,030,199, Trick et al., in U.S. Pat. No. 5,114,398, and Flinchbaugh, in U.S. Pat. No. 6,673,051 all describe an external valve for an indwelling urinary catheter that is actuated by a magnet.

The valves disclosed in these patents are opened whenever a magnet is placed next to the external valve. However, unintended discharge can result if a magnet accidentally comes into close contact with the valve or if the patient inadvertently crosses a magnet field. These valves are also relatively complex, difficult to manufacture, and expensive.

Most external valves for indwelling urinary catheters in the prior art are actuated by palpitation. Whenever the patient feels the need to urinate, an external valve is squeezed or a button on the valve is pressed. These particular external valves can take a variety of shapes and require different methods of palpitation. For example, Haber et al., in U.S. Pat. No. 4,813,935, Goldberg et al., in U.S. Pat. No. 5,234,409, Salama, in U.S. Pat. No. 5,306,226, Mikhail et al., in U.S. Pat. Nos. 5,707,357 and 6,050,934, Polyak et al., in U.S. Pat. No. 6,056,687, Deininger et al., in U.S. Pat. No. 6,200,261, and Beufore et al., in U.S. Pat. No. 7,037,303 all describe an external valve for an indwelling urinary catheter that is actuated by squeezing a bulb, dome, or tube. These external valves remain closed until the patient squeezes the valve causing deformation of the structure by collapsing the walls of the valve, thereby providing an opening through which urine flows out of the patient. Palpable external valves of this type can leak and can lose effectiveness over the long run as constant squeezing can eventually cause permanent structural deformation. Another version of the palpable external valve for indwelling catheters is actuated whenever the patient compresses the valve by squeezing flaps or sliding a moveable segment . External valves with squeezable flaps or moveable segments can be found in Staeubli U.S. Pat. No. 4,570,898, Cross et al. U.S. Pat. No. 5,135,199, Gigante U.S. Pat No. 5,445,626, and Pfeiderer et al. U.S. Pat. No. 6,106,503. In yet another type of externally palpable valve for urinary indwelling catheters, Schonbachler et al. in U.S. Pat. No. 5,522,806, describe an external valve that is actuated whenever the patient presses a button. These external valves are complex, unreliable, susceptible to leakage, easily actuated accidentally, difficult to make, expensive and require proficient manual dexterity.

Other types of external palpable valves for indwelling urinary catheters are actuated by rotation. In these valves, from Berry et al. in U.S. Pat. No. 5,158,553 and Eschel in U.S. Pat. No. 6,270,053, when the patient rotates a segment of the valve in one direction, the valve opens, and when the valve is rotated in the opposite direction the valve closes.

The prior art also includes an external valve that is actuated by fluid pressure. Parsons et al. in U.S. Pat. No. 4,424,058 describe an external pressure relief valve that opens whenever a predetermined fluid pressure is reached. This valve is bulky, complex, expensive, susceptible to leaks, and may not be appropriate for all patients.

### SUMMARY OF THE INVENTION

The present invention describes a unique external urethral valve that closes reliably at both high and low pressures, drains completely, can be connected to a collection bag when necessary for immobile or incapacitated patients, and will not accidently discharge, can be left open for a collection bag, is easy to manipulate, is not bulky, is inexpensive, is simple, has a low risk of infection, is inconspicuous, and is compatible to an individual's normal daily activities.

The external urethral valve of the present invention can be connected to any indwelling urethral catheter, such as a Foley, or Foley-type, catheter and is opened, or actuated when the patient squeezes the valve cap against a flange attached to the valve body. The valve of the present invention consists of a stem with a hollow portion which forms a conduit to the outside and an opening that is sealed against the walls of the valve body with the use of a compression spring. The spring keeps the valve closed by keeping the moveable cap extended away from the body of the patient and sealing the hole (in the stem) against the valve seat of the valve body, thereby preventing urine from flowing into the hole of the stem which communicates with the hollow portion that exits to the outside. When urination is required, the patient simply squeezes the cap of the valve towards a flange, thereby compressing the spring, pushing the hollow stem forward, opening the hole in the stem, and allowing urine to flow through the hole in the valve into a collection bag or appropriate waste receptacle.

The valve of the present invention has advantages over other valves in the prior art in that it does not require implantation procedures (a requirement for all internal valves), and is actuated by squeezing the moveable section towards the patient, as opposed to external valves that are opened by pulling a moveable piece away from the patient, a process which could lead to the patient accidently tugging the urethral catheter out of its position within the body.

### BRIEF DESCRIPTION OF THE DRAWINGS

FIG. 1 shows a perspective view of the preferred embodiment of the external urethral valve.

FIG. 2 shows a longitudinal cross section of the external urethral valve in the closed position.

FIG. 3 shows a longitudinal cross section of the external urethral valve in the open position.

FIG. 4 shows a longitudinal cross section of the stem of the external urethral valve.

FIG. 5 shows the stem of the external urethral valve with an alternate opening embodiment.

### DETAILED DESCRIPTION OF THE PREFERRED EMBODIMENT

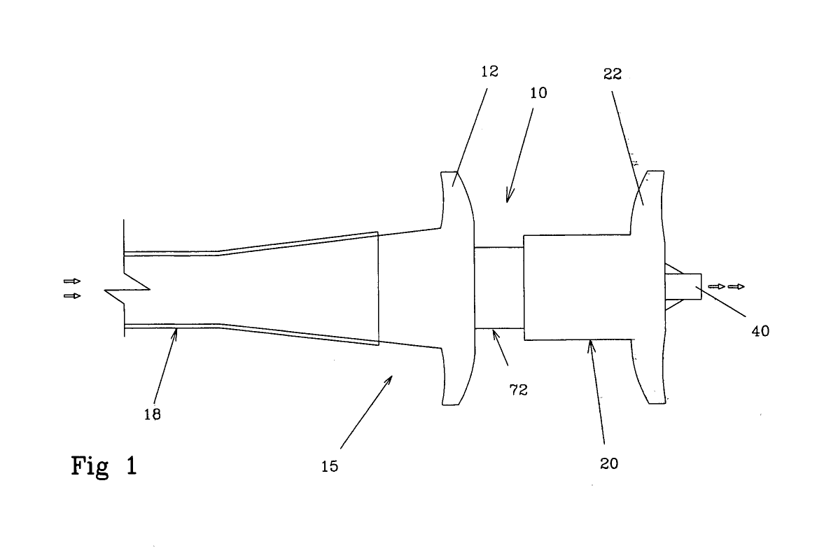

FIG. 1 depicts the preferred embodiment of the external urethral valve (10). The external urethral valve (10) includes a hollow adapter section (15), a distal end of which is sized and configured to frictionally engage within a proximal end of a standard indwelling urethral catheter (18) as illustrated. The valve (10) also includes a moveable cap (20) integrating a valve stem (40) therein which terminates in a valve head (48) positioned within the hollow adapter section (15), the valve stem (40) extending into and through the body of the adapter section (15) through a proximal bore (80) therein. When the cap (20) is squeezed or moved against a flange (12) of the hollow adapter section (15) using a flange (22) on the cap (20) whenever the user wants to open the valve (10) in order to empty the bladder. Once the cap (20) is moved toward the hollow adapter section (15) along a hollow neck portion (72) through which a hollow valve stem (40) extends, and around which is seated a biasing spring (30), the valve (10) will open, and urine will exit through the opening (25) in the cap (20), as will be described in greater detail hereinafter. The valve (10) will completely close once pressure is no longer exerted on the cap (20) under force of the biasing spring (30). The hollow valve stem (40) is engaged to the cap (20) by any known suitable means, such as by gluing.

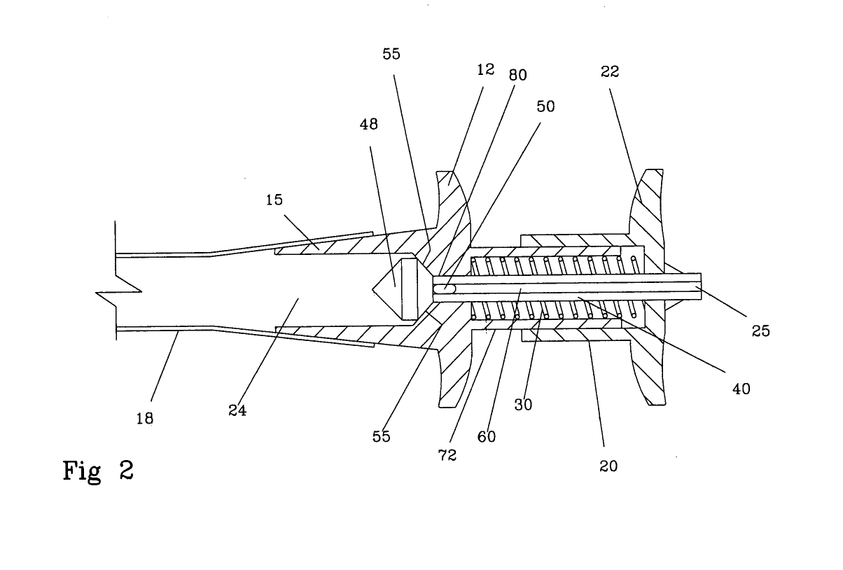

FIG. 2 shows an interior longitudinal cross section of the external urethral valve (10) when the valve (10) is in the closed position. The hollow valve stem (40) extends through the hollow neck portion (72) and proximal bore (80) within the adapter section (15) and terminates in a valve head (48) which abuts against internal proximal shoulders or valve seat (55) of the adapter section (15) of the valve (10) and is maintained thereagainst through the use of the biasing spring (30). The biasing spring (30) maintains the valve (10) in the closed position. The valve stem (40) has a radial opening (50), just proximal to the valve head (48), which communicates with a longitudinal bore (60) extending through the valve stem 40 and into communication with the opening 25 located at a proximal end of the valve (10). As long as the spring (30) maintains the valve head (48) of the valve stem (40) pressed against the valve seat (55), the opening (50) remains sealed, preventing urine from flowing into opening (50) and bore (60) and out of the proximal opening (25) of the valve (10).

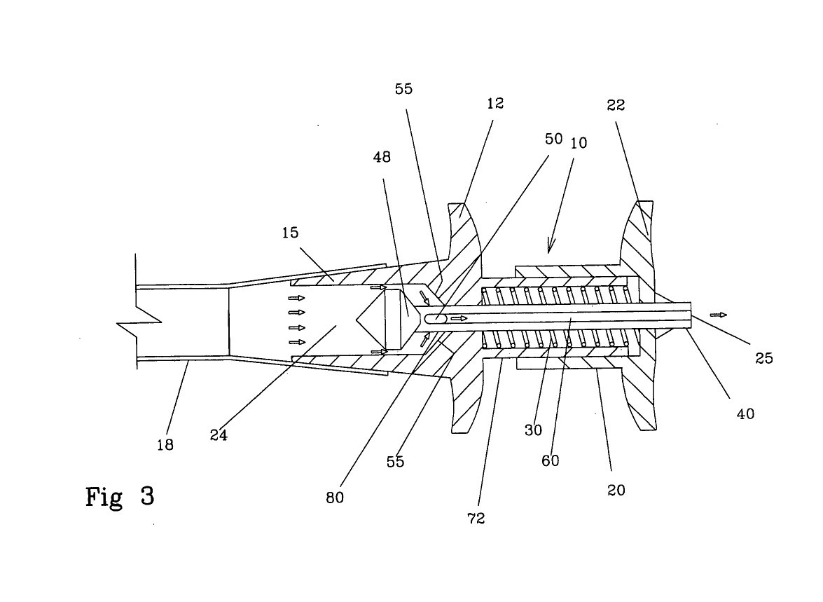

FIG. 3 shows an interior longitudinal cross section of the external urethral valve (10) when the valve (10) is in the open position. When a user compresses the cap section (20) toward the adapter section (15) with the aid of the flanges (12) and (22), the spring (30) is compressed, and the hollow valve stem (40) is pushed into a distal hollow area (24) of the adapter (15), off the valve seat (55). Once the valve stem (40) is thusly positioned, the opening (50) located proximally adjacent the head (48) of the stem (40) is moved into communication with the distal hollow interior area (24) of the adapter (15) opening a path through which urine flows, into the bore (60), through the hollow valve stem (40), and out through the proximal opening (25) in the valve stem (40).

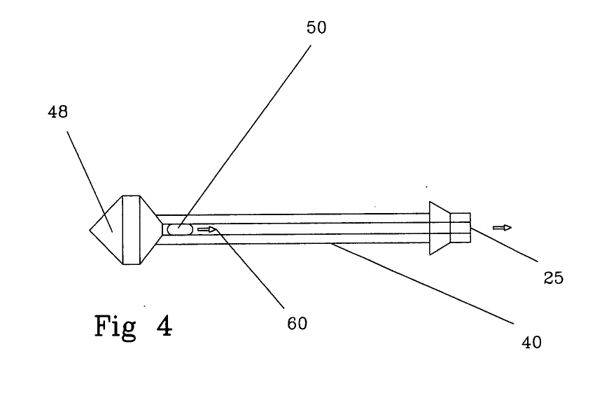

FIG. 4 shows a first embodiment of the hollow valve stem (40) of the external urethral valve (10). The opening (50), in fluid communication with bore (60) extending through the valve stem (40) thereof, located at a position adjacent the valve head (48) of the valve stem (40), allows urine to flow through the stem (40), and out of the valve (10) through the proximal opening (25), when the valve (10) is actuated or opened by the user as described above.

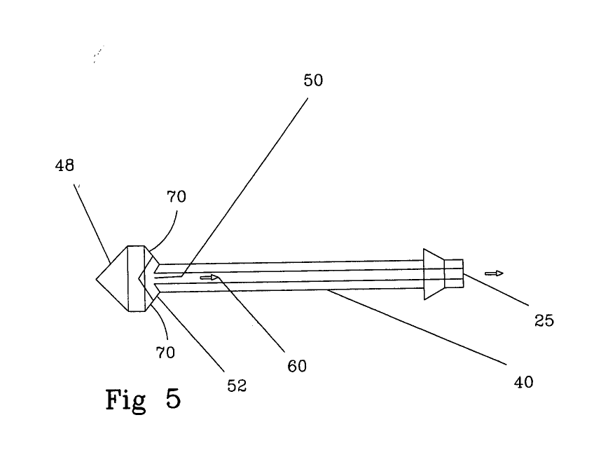

FIG. 5 shows an alternate embodiment for the opening (50) wherein it extends into the head (48) of the valve stem (40) and two lateral bores (52) are provided in a rear edge (70) of the valve head (48), which seats against valve seat (55) when the valve (10) is closed and is moved off the valve seat (55) by compressing the cap (20) toward the adapter (15), for the fluid to pass through, and into the bore (60) through the valve stem (40) and out the opening (25).

We claim:

1. A external urinary control valve comprising;
a hollow adapter having distal and proximal open ends defining a passageway therethrough, a distal end of which engages an indwelling catheter;
a hollow stem extending into and through the open proximal end of the hollow adapter, a valve head of the stem seating within the hollow at a distal end of the adapter for providing a normally closed passageway for urine discharge;
a movable cap attached to the said hollow stem, which controls opening of the normally closed passageway;
the valve head at the distal end of the hollow stem seating against inwardly directed shoulders of the hollow adapter when biased thereagainst;
the hollow stem further having at least one opening therein which is in fluid communication with the passageway; and
a biasing spring which maintains the valve in a closed position.

2. The external urinary control valve of claim 1, wherein:
the opening in the hollow stem is located proximal to the valve head.

3. The external urinary control valve of claim 1, wherein:
the adapter is sized and configured to receive and frictionally hold a proximal end of an indwelling catheter over a distal end of the adapter.

4. The external urinary control valve of claim 1, wherein:
the opening in the head of the hollow valve stem comprises at least one laterally extending bore extending from a rear edge of the head into communication with the passageway in the valve stem.

5. The external urinary control valve of claim 1 wherein:
the adapter includes a hollow cylinder at a proximal end thereof through which the hollow valve stem extends and about which is positioned a biasing spring which pushes proximally against the cap to maintain the valve in a closed position.

6. The external urinary control valve of claim 5, wherein:
the cap is positioned around and over an open proximal end of the hollow cylinder of the adapter.

7. The external urinary control valve of claim 6, wherein:
the cap is provided with laterally extending flanges and the adapter is also provided with laterally extending flanges.

8. The external urinary control valve of claim 7, wherein:
the valve is opened by pushing the flanges on the cap toward the flanges on the adapter.

9. The external urinary control valve of claim 8, wherein:
the valve is closed by expansion of the biasing spring when pressure on the flanges is released.

10. An external urinary control valve for allowing flow of urine through a passageway therethrough extending from a proximal end of a standard indwelling catheter comprising:
a distal catheter engaging portion and a proximal moveable portion, the portions defining a passageway therethrough from the distal end of the catheter to an exit at the proximal end of the proximal moveable portion;
the portions being biased away from one another to maintain the valve in a closed position;
and means for moving the proximal moveable portion toward the distal catheter engaging portion to open the valve and allow for flow of urine outwardly therefrom.
